(19) United States
(12) Patent Application Publication
(10) Pub. No.: US 2007/0250079 A1
(43) Pub. Date: Oct. 25, 2007

(54) Dermabrasive tool and method of use

(76) Inventor: Dagmar Kern, Chantilly, VA (US)

Correspondence Address: Leander F. Aulisio, 320 23rd Street #705, Arlington, VA, 22202, US

(21) Appl. No.: 11/407,981

(22) Filed: Apr. 21, 2006

(51) Int. Cl. A61B 17/50

(52) U.S. Cl. 606/131

(57) ABSTRACT

A lightweight hand-held tool useful for performing cosmetic dermabrasion comprising a handle of aluminum tubing, a flattened head of aluminum, at least one removable pad attached to the flattened head and an attachment means for removably connecting the removable pad to the flattened head; wherein the attachment means covers the flattened head and comprises hook and loop fasteners for attachment to the removable pad that also contains hook and loop fasteners.

### BACKGROUND OF THE INVENTION

1. Field of the Invention

The present invention relates to a method for cleaning and removing dead and calloused skin on various body parts. The body parts can be the foot, the hand, the elbow or the knee. Preferably, the body part is the foot. A lightweight hand-held tool is also disclosed for practicing the method.

2. Discussion

The skin is an organ that functions as a barrier to the outside environment. It comprises three integrated layers that are known as the epidermis, the dermis and the subcutis. The outermost layer of the skin is the epidermis. The epidermis has immediate exposure to the outside environment and offers the greatest protection to the organism. The lowermost layer of the epidermis is known as the basal layer. The basal layer is where new skin cells are being constantly generated as by cell division. The new cells form a continuous layer that can ascend toward the surface of the epidermis as an intact layer. This mechanism of the organism allows for continual replacement of the epidermis, and thus continual fortification of the most important barrier layer to the outside environment.

The uppermost part of the epidermis is known as the stratum corneum. Another name for the stratum corneum is the horny corneal layer. This layer comprises a substantial number of dead skin cells. Also excess dead skin cells and sebaceous oils easily accumulate on the stratum corneum. The sebum, or sebaceous oils, are fatty acids that tend to increase the acidity of the skin. The skin of the foot, the hand and the elbow tends to accumulate an oversized and very rough horny corneal layer.

The skin constantly confronts a vast array of flora and fauna that attempt to establish a foothold somewhere on the skin. Various areas of the skin are more prone to attack by certain microbes than other areas. Even apparently clean skin can have millions of living organisms on its surface. Many of these microbes are substantially permanent members that vary from one region of the body to another. Bacteria, mites and yeast all seek to find a friendly environment somewhere on the skin in which to survive, grow and propagate. Also bacteria and other organisms can easily spread from one person to another whenever there is physical contact of any kind.

The skin has a tendency to accumulate detritus such as excess dead skin, dander and flakes. When this happens, the skin finds it difficult to perform its function of assisting in the regulation of the body's temperature. Also, dirty and oily skin is more susceptible to attack by various microbes. The presence of scrapes or cuts on the skin is more critical when the skin is not clean, allowing for ready invasion of viruses and bacteria into the organism. If the skin of the organism is unclean, the pores of the skin become clogged with sebum and detritus; and the skin becomes less effective in protecting the organism from the environment.

As a human organism ages, the skin of the organism typically becomes less elastic and more dry. The risk of skin problems increases with a dry and inelastic skin. A youthful skin is not only aesthetically pleasing; it is also more healthy. In order for an aging human organism to retain that youthful-looking skin, it is necessary that the skin of the human organism be periodically cleaned as by cosmetic dermabrasion to remove such elements as flora, fauna, dead cells, sebum and the like from the skin. This is preferably done by a professional dermatologist.

U.S. Pat. No. 6,290,707, issued to Street, relates to a cosmetic abrasive pad for use in scrubbing a section of the skin to remove detritus. The abrasive pad can be in the form of a flexible milt having an opening to receive the user's hand. The abrasive pad can, in the alternative, be a handpiece comprising a substantially rigid elongate handle having a proximal end and a distal end. The distal end carries a segment of mixed denier filaments that are lofty and fibrous, and further contain biocompatible abrasive particles. See FIG. 14 for an illustration of the handpiece, which is much like a tongue depressor. The handpiece is preferably made of plastic or wood. See Column 10, line 42.

U.S. Pat. No. 5,997,549, issued to Sauceda et al, relates to a tool for removing warts, the tool comprising an elongated handle and a metallic pad affixed to the handle. The pad is constructed of carbide that includes cobalt. A method of removing warts is also disclosed.

U.S. Pat. No. 5,560,068, issued to Blake, relates to a foot care shower brush comprising a handle, a cylindrical brush comprising a mounting rod extending from the handle, and a plurality of radially extending nylon bristless projecting outwardly from the rod.

U.S. Pat. No. 3,196,885, issued to Emerson, relates to a file for removing corns and calluses. The file comprises a handle, a working element connected to the handle, and a coating of abrasive material covering the working element.

U.S. Pat. No. 3,131,701, issued to Emerson, relates to a file for removing corns and calluses. The file comprises a flaring spatulate core terminated in a rounded end. Various areas of the core have either a convex or concave shape. A hardened mass of material is baked on the spatulate core. The mass includes a comminuted abrasive material. A handle is positioned at the narrower end of the core.

U.S. Pat. No. 3,198,198, issued to Bittner, relates to a corn and callous file comprising a handle, a skeleton frame, and two apertured cutting members. The two cutting members are secured over opposed sides of the skeleton frame.

U.S. Pat. No. 2,898,914, issued to Sardal, relates to a foot rasper comprising a handle, a body portion having a dovetail-shaped groove, a rasp piece of thin springy sheet material designed to be pushed into the groove, and a flat spring carried by the handle.

U.S. Pat. No. 132,468, issued to Jacobsohn, relates to an instrument for removing corns and calluses comprising a blade or rasping portion.

A number of Design Patents are also disclosed as prior art: D 431,096, issued to Rieser; D 442,741, issued to Rieser; D 486,607, issued to Chien; and D 496,731, issued to Park. All disclose various designs for manicure and pedicure products.

U.S. Pat. No. 6,290,702, issued to Street, relates to a cosmetic abrasive pad for use in scrubbing a section of the skin to remove detritus. The abrasive pad can be in the form of a flexible mitt having an opening to receive the user's hand. The abrasive pad can, in the alternative, be a handpiece comprising a substantially rigid elongate handle having a proximal end and a distal end. The distal end carries a segment of mixed denier filaments that are lofty and fibrous, and further contain biocompatible abrasive particles. See FIG. 14 for an illustration of the handpiece, which is much like a tongue depressor. The handpiece is preferably made of plastic or wood. See Column 10, line 42.

U.S. Pat. No. 5,997,549, issued to Sauceda et al, relates to a tool for removing warts, the tool comprising an elongated handle and a metallic pad affixed to the handle. The pad is constructed of carbide that includes cobalt. A method of removing warts is also disclosed.

U.S. Pat. No. 5,560,068, issued to Blake, relates to a foot care shower brush comprising a handle, a cylindrical brush comprising a mounting rod extending from the handle, and a plurality of radially extending nylon bristles projecting outwardly from the rod.

U.S. Pat. No. 3,196,885, issued to Emerson, relates to a file for removing corns and calluses. The file comprises a handle, a working element connected to the handle, and a coating of abrasive material covering the working element.

U.S. Pat. No. 3,131,701, issued to Emerson, relates to a file for removing corns and calluses. The file comprises a flaring spatulate core terminated in a rounded end. Various areas of the core have either a convex or concave shape. A hardened mass of material is baked on the spatulate core. The mass includes a comminuted abrasive material. A handle is positioned at the narrower end of the core.

U.S. Pat. No. 3,198,198, issued to Bittner, relates to a corn and callous file comprising a handle, a skeleton frame, and two apertured cutting members. The two cutting members are secured over opposed sides of the skeleton frame.

U.S. Pat. No. 2,898,914, issued to Sardal, relates to a foot rasper comprising a handle, a body portion having a dovetail-shaped groove, a rasp piece of thin springy sheet material designed to be pushed into the groove, and a flat spring carried by the handle.

U.S. Pat. No. 132,468, issued to Jacobsohn, relates to an instrument for removing corns and calluses comprising a blade or rasping portion.

A number of Design Patents are also disclosed as prior art: D 431,096, issued to Rieser; D 442,741, issued to Rieser; D 486,607, issued to Chien; and D 496,731, issued to Park. All disclose various designs for manicure and pedicure products.

### SUMMARY OF THE INVENTION

A hand-held tool is disclosed. The tool is useful in the performance of dermabrasion. In a preferred embodiment, the dermabrasion is performed by a professional cosmetologist or manicurist. The skin is completely dried before the dermabrasion is performed. The tool comprises a handle comprising an elongated substantially cylindrical lightweight metal object. The tool further comprises a flattened head having two sides. The flattened head is comprised of the same lightweight metal as the handle. Preferably, the tool is made from one piece of lightweight metal tubing; the piece having a distal end and a proximal end. The distal end comprises the flattened head and the proximal end comprises the substantially cylindrical handle. Preferably, the tool is prepared by a process comprising: obtaining a cylindrical piece of lightweight metal tubing, the cylindrical piece being hollow in the middle and having open ends. The piece of metal is then flattened at one end as by pressing about 40% of the length of the piece with a hydraulic press or the like. The remaining about 60% of the metal piece is employed as the handle.

The cylindrical piece of lightweight metal tubing is preferably about ½ inch to about 1 and ½ inches in diameter. These dimensions allow for the tool to be readily held by a human hand. The wall thickness of the metal tubing is about 1 inch to about 1.3 inches. A removable flexible cushioned covering is employed to provide a user with a comfortable grip. Preferably the covering overlays substantially the entire handle. In a preferred embodiment, the covering is prepared from a material comprising a vinyl polymer. It is within the scope of the invention to provided a covering prepared from other polymeric materials, be they synthetic or natural or mixtures of both. The covering for the handle is made removable so that the entire metallic part of the tool can be easily sterilized and then reassembled.

The hand-held tool further comprises at least one removable pad for dermabrasion. The pad has two sides. A primary side contains an abrasive material and a secondary side contains hook and loop fasteners. The secondary side attaches to a web of material useful as an attachment means. The attachment means comprises a web for covering the flattened head. The web has two sides. A first side contains an adhesive material and a second side contains hook and loop fasteners. In a preferred embodiment, the attachment means contacts the flattened head of the tool on both sides of the head. Preferably, this is accomplished by attaching half of the web to one side of the flattened head, pulling the remainder of the web over the top of the head, and attaching the second half of the web to the other side of the flattened head. The adhesive material can be any of the known materials for bonding metal to plastic, and as such forms no part of the present invention. The adhesive material can form a permanent bond between metal and non-metal; or, in another embodiment, the adhesive material can be such that the bond formed is non-permanent, allowing for separation of the attachment means from the flattened metallic head.

The dimensions of the attachment means are preferably substantially the same as the dimensions of the flattened head. Preferably, the shape of the metallic head is substantially rectangular. The dimensions of the flattened head are preferably about 2 inches by about 3 and ½ inches.

Since the flattened head is covered by the attachment means on both sides of the head, more than one removable pad can be employed if so desired. Two pads, one on each side of the flattened head, can be joined to the attachment means as by hook and loop fasteners. This allows for easy removal when the dermabrasion is completed. Each of the removable pads comprises an abrasive material. The grade of the abrasive material can be the same for both pads, or it can be different. The tool containing two pads of different grades of abrasive material is more versatile. Both coarse work and fine work can be performed with a tool that has two different grades of abrasive material, one pad having coarse ground material and a second pad having fine ground material. In a preferred embodiment, the abrasive material is silicon carbide. One skilled in the art can substitute any other material that can perform the function of an abrasive. The particular type of abrasive material does not form any part of the invention, and as such does not impart any limitation to the present inventive concept, unless otherwise stated or implied.

In a preferred embodiment, the lightweight metal is aluminum. An aluminum tool is easy to handle, yet strong enough to resist fracture when pressure is exerted. Plastic and wood do not have these advantages. Aluminum metal is also easy to sterilize. It also is very durable, allowing for extended use.

### BRIEF DESCRIPTION OF THE DRAWINGS

FIG. 1 is an illustration of the lightweight metal part of the cosmetic tool, showing the handle and the flattened head.

FIG. 2 is an illustration of the lightweight metal part of the cosmetic tool covered with the attachment means. The attachment means comprises a web of material having two sides. The first side containing an adhesive material is in contact with the flattened head of the metal part of the tool; and the second side contains hook and loop fasteners for attaching an abrasive pad to the cosmetic tool.

FIG. 3 is an illustration of the complete cosmetic tool comprising a metal handle, a flattened metal head, an attachment means, at least one abrasive pad and a handle covering.

FIG. 4 is an illustration of a side view of a cosmetic tool comprising a metal handle, a flattened metal head, two abrasive pads, an attachment means for connecting the abrasive pads to the flattened metal head, and a non-slip handle.

FIG. 5 is an illustration of a top view of the complete cosmetic tool.

FIG. 6 is an illustration of an abrasive pad useful for dermabrasion displaying a Velcro™ side and a side comprising abrasive particles.

### DETAILED DESCRIPTION OF THE INVENTION

A hand-held tool is disclosed. The tool is useful in the performance of dermabrasion. In a preferred embodiment, the dermabrasion is performed by a professional cosmetologist or manicurist. The skin is completely dried before the dermabrasion is performed. The tool comprises a handle comprising an elongated substantially cylindrical lightweight metal object. The tool further comprises a flattened head having two sides. The flattened head is comprised of the same lightweight metal as the handle. Preferably, the tool is made from one piece of lightweight metal tubing; the piece having a distal end and a proximal end. The distal end comprises the flattened head and the proximal end comprises the substantially cylindrical handle. Preferably, the tool is prepared by a process comprising: obtaining a cylindrical piece of lightweight metal tubing, the cylindrical piece being hollow in the middle and having open ends. The piece of metal is then flattened at one end as by pressing about 40% of the length of the piece with a hydraulic press or the like. The remaining about 60% of the metal piece is employed as the handle.

The cylindrical piece of lightweight metal tubing is preferably about ½ inch to about 1 and ½ inches in diameter. These dimensions allow for the tool to be readily held by a human hand. The wall thickness of the metal tubing is about 1 inch to about 1.3 inches. A removable flexible cushioned covering is employed to provide a user with a comfortable grip. Preferably the covering overlays substantially the entire handle. In a preferred embodiment, the covering is prepared from a material comprising a vinyl polymer. It is within the scope of the invention to provided a covering prepared from other polymeric materials, be they synthetic or natural or mixtures of both. The covering for the handle is made removable so that the entire metallic part of the tool can be easily sterilized and then reassembled.

The hand-held tool further comprises at least one removable pad for dermabrasion. The pad has two sides. A primary side contains an abrasive material and a secondary side contains hook and loop fasteners. The secondary side attaches to a web of material useful as an attachment means. The attachment means comprises a web for covering the flattened head. The web has two sides. A first side contains an adhesive material and a second side contains hook and loop fasteners. In a preferred embodiment, the attachment means contacts the flattened head of the tool on both sides of the head. Preferably, this is accomplished by attaching half of the web to one side of the flattened head, pulling the remainder of the web over the top of the head, and attaching the second half of the web to the other side of the flattened head. The adhesive material can be any of the known materials for bonding metal to plastic, and as such forms no part of the present invention. The adhesive material can form a permanent bond between metal and non-metal; or, in another embodiment, the adhesive material can be such that the bond formed is non-permanent, allowing for separation of the attachment means from the flattened metallic head.

The dimensions of the attachment means are preferably substantially the same as the dimensions of the flattened head. Preferably, the shape of the metallic head is substantially rectangular. The dimensions of the flattened head are preferably about 2 inches by about 3 and ½ inches.

Since the flattened head is covered by the attachment means on both sides of the head, more than one removable pad can be employed if so desired. Two pads, one on each side of the flattened head, can be joined to the attachment means as by hook and loop fasteners. This allows for easy removal when the dermabrasion is completed. Each of the removable pads comprises an abrasive material. The grade of the abrasive material can be the same for both pads, or it can be different. The tool containing two pads of different grades of abrasive material is more versatile. Both coarse work and fine work can be performed with a tool that has two different grades of abrasive material, one pad having coarse ground material and a second pad having fine ground material. In a preferred embodiment, the abrasive material is silicon carbide. One skilled in the art can substitute any other material that can perform the function of an abrasive. The particular type of abrasive material does not form any part of the invention, and as such does not impart any limitation to the present inventive concept, unless otherwise stated or implied.

In a preferred embodiment, the lightweight metal is aluminum. An aluminum tool is easy to handle, yet strong enough to resist fracture when pressure is exerted. Plastic and wood do not have these advantages. Aluminum metal is also easy to sterilize. It also is very durable, allowing for extended use.

A hand-held tool is disclosed. The tool is useful in the performance of dermabrasion. In a preferred embodiment, a professional cosmetologist or manicurist performs the dermabrasion. The skin is completely dried before the dermabrasion is performed. The tool comprises a handle comprising an elongated substantially cylindrical lightweight metal object. The tool further comprises a flattened head having two sides. The flattened head is comprised of the same lightweight metal as the handle. Preferably, the tool is made from one piece of lightweight metal tubing, the piece having a distal end and a proximal end. The distal end comprises the flattened head and the proximal end comprises the substantially cylindrical handle. Preferably, the tool is prepared by a process comprising: obtaining a cylindrical piece of lightweight metal tubing, the cylindrical piece being hollow in the middle and having open ends. The piece of metal is then flattened at one end as by pressing about 40% of the length of the piece with a hydraulic press or the like. The remaining about 60% of the metal piece is employed as the handle.

The cylindrical piece of lightweight metal tubing is preferably about ½ inch to about 1 and ½ inches in diameter. These dimensions allow for the tool to be readily held by a human hand. The wall thickness of the metal tubing is about 1 inch to about 1.3 inches. A removable flexible cushioned covering is employed to provide a user with a comfortable grip. Preferably the covering overlays substantially the entire handle. In a preferred embodiment, the covering is prepared from a material comprising a vinyl polymer. It is within the scope of the invention to provided a covering prepared from other polymeric materials, be they synthetic or natural or mixtures of both. The covering for the handle is made removable so that the entire metallic part of the tool can be easily sterilized and then reassembled.

The hand-held tool further comprises at least one removable pad for dermabrasion. The pad has two sides. A primary side contains an abrasive material and a secondary side contains hook and loop fasteners. The secondary side attaches to a web of material useful as an attachment means. The attachment means comprises a web for covering the flattened head. The web has two sides. A first side contains an adhesive material and a second side contains hook and loop fasteners. In a preferred embodiment, the attachment means contacts the flattened head of the tool on both sides of the head. Preferably, this is accomplished by attaching half of the web to one side of the flattened head, pulling the remainder of the web over the top of the head, and attaching the second half of the web to the other side of the flattened head. The adhesive material can be any of the known materials for bonding metal to plastic, and as such forms no part of the present invention. The adhesive material can form a permanent bond between metal and non-metal; or, in another embodiment, the adhesive material can be such that the bond formed is non-permanent, allowing for separation of the attachment means from the flattened metallic head.

The dimensions of the attachment means are preferably substantially the same as the dimensions of the flattened head. Preferably, the shape of the metallic head is substantially rectangular. The dimensions of the flattened head are preferably about 2 inches by about 3 and ½ inches.

Since the flattened head is covered by the attachment means on both sides of the head, more than one removable pad can be employed if so desired. Two pads, one on each side of the flattened head, can be joined to the attachment means as by hook and loop fasteners. This allows for easy removal when the dermabrasion is completed. Each of the removable pads comprises an abrasive material. The grade of the abrasive material can be the same for both pads, or it can be different. The tool containing two pads of different grades of abrasive material is more versatile. Both coarse work and fine work can be performed with a tool that has two different grades of abrasive material, one pad having coarse ground material and a second pad having fine ground material. In a preferred embodiment, the abrasive material is silicon carbide. One skilled in the art can substitute any other material that can perform the function of an abrasive. The particular type of abrasive material does not form any part of the invention, and as such does not impart any limitation to the present inventive concept, unless otherwise stated or implied.

In a preferred embodiment, the lightweight metal is aluminum. An aluminum tool is easy to handle, yet strong enough to resist fracture when pressure is exerted. Plastic and wood do not have these advantages. Aluminum metal is also easy to sterilize. It also is very durable, allowing for extended use.

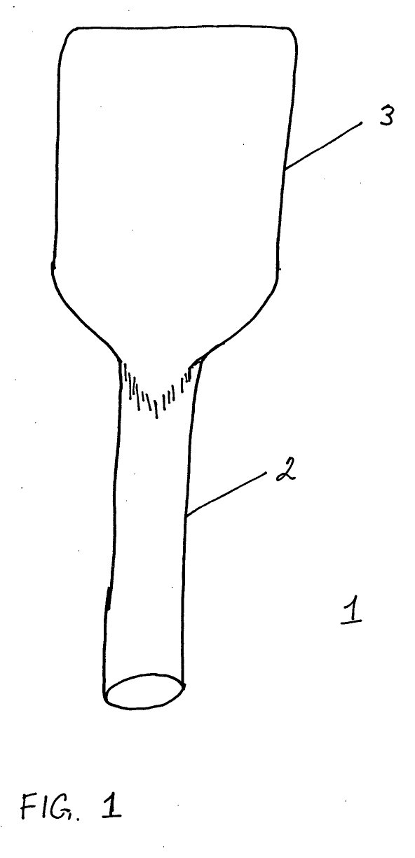

Referring now to the Figures, FIG. 1 is an illustration of the lightweight metal tool 1 having a cylindrical handle 2 and a flattened head 3. The lightweight metal is preferably aluminum metal. The aluminum metal can be readily cleaned and sterilized for continued use. The flattened head can be easily formed as by pressing an end of a cylindrical piece of aluminum tubing with a hydraulic press or the like.

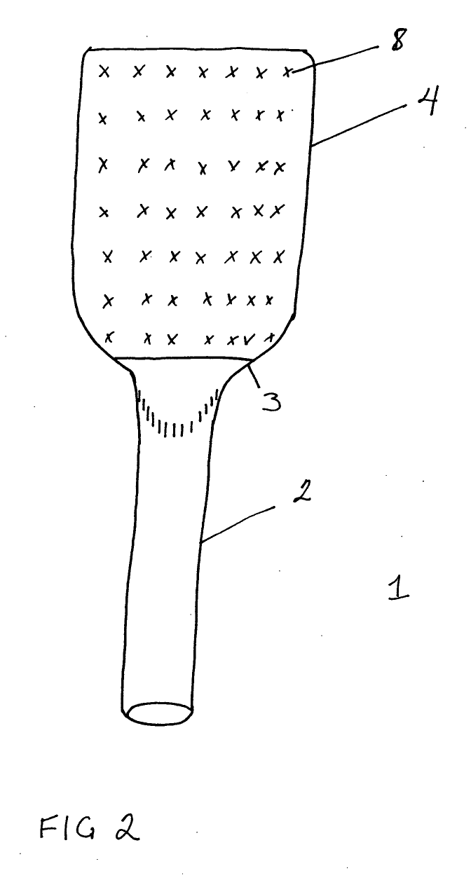

FIG. 2 is an illustration of a lightweight metal tool 1 wherein the flattened head 3 of the tool 1 is covered with an attachment means 4 for covering both sides of the flattened head 3. Hook and loop fasteners (Velcro™) 8 are located on the outwardly facing side of the attachment means 4. The attachment means 4 is firmly secured to the flattened head 3 by means of adhesive material. The adhesive material can be either a permanent sealing material or a removably sealable material. The cylindrical handle 2 is an extension of the flattened head 3.

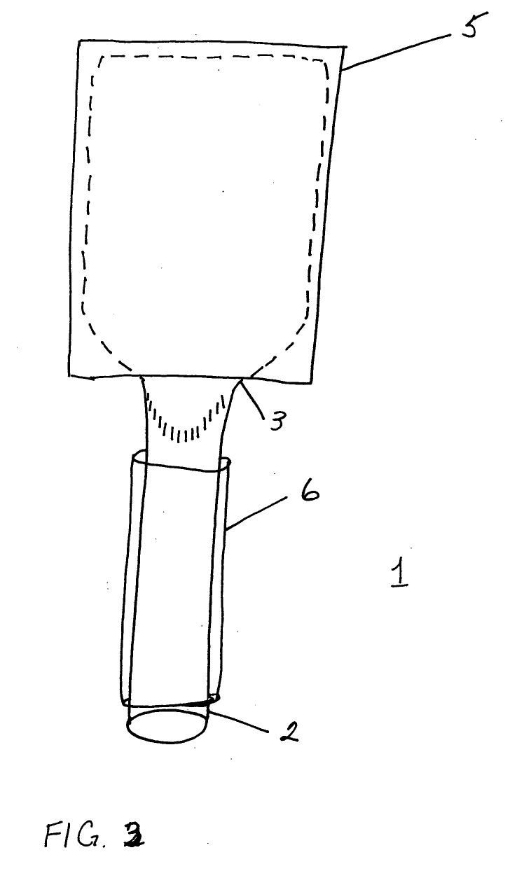

FIG. 3 is an illustration of a lightweight metal tool 1 wherein the handle 2 is covered by a flexible cushioned handpiece covering 6. Preferably, the covering 6 is made of a vinyl material. The covering 6 for the handle 2 is easily removable for cleaning and the like. The covering 6 can be of any design, and can include ridges and grooves for improved gripping by the human hand. The flattened head 3 is covered by a removable pad 5 for dermabrasion. Between the flattened head 3 and the pad 5 is the attachment means 4 (not shown). The removable pad 5 comprises abrasive particles on the surface of the pad 5 that is exposed to the environment. The surface of pad 5 that connects to the attachment means 4 comprises hook and loop fasteners (Velcro™) that join with the Velcro™ of the attachment means 4 to form a secure bond.

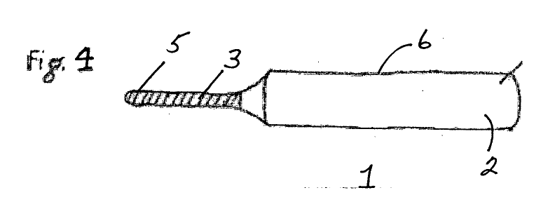

FIG. 4 is an illustration of a sideview of a lightweight metal tool 1 wherein the handle 2 is covered by a flexible cushioned non-slip grip cover 6. The cover 6 can overlap the handle 2 completely, as in this figure. The flattened head 3 is covered by a removable pad 5 for dermabrasion. The removable pad 5 can cover the flattened head 3 in a complete fashion and on both sides of the head 3, as in this illustration. Between the flattened head 3 and the pad 5 is an attachment means (not shown). The attachment means can be a single piece of fabric material that covers one side of the flattened head 3, wraps over the top of the head 3 and then covers the second side of the head 3. The attachment means is preferably a piece of fabric that consists of two sides, a first side comprising an adhesive material for contacting the aluminum metal of the flattened head 3; and a second side comprising Velcro™ for attachment to a removable pad 5.

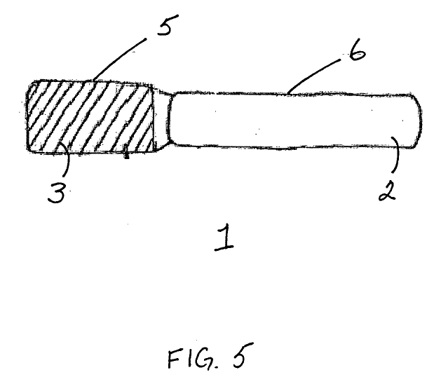

FIG. 5 is an illustration of a top view of the lightweight metal tool 1. The handle 2 is covered by a flexible cushioned non-slip grip cover 6. The flattened head 3 is covered by a removable pad 5 for dermabrasion. Between the flattened head 3 and the pad 5 is the attachment means (not shown). A second removable pad can be positioned on the back side of the flattened head 3 (not shown). The removable pad 5 comprises a plurality of abrasive particles in a homogeneous arrangement on the exposed surface of the pad 5. The abrasive particles are suitable for dermabrasion. In a preferred embodiment, a first removable pad on one side of the tool comprises a course grade of abrasive particles and a second removable pad on the other side of the tool comprises a fine grade of abrasive particles. The surface of removable pad 5 that connects the pad 5 to the attachment means comprises Velcro™ for removable connection.

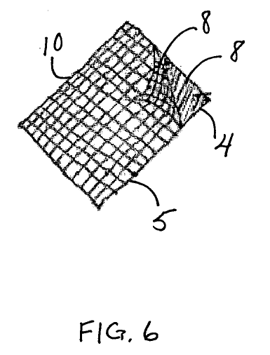

FIG. 6 is an Illustration of removable pad 5 and attachment means 4. Removable pad 5 can be made of a material that is woven or nonwoven. The outer surface of pad 5 comprises abrasive particles, the particles being acceptable for dermatological use. The inner surface of pad 5 contains Velcro™ 8. Attachment means 4 contains Velcro™ 8 on its surface that directly contacts the removable pad 5. On the other surface of the attachment means 4 is an adhesive composition (not shown). The adhesive composition can be of a permanent bonding nature or of a temporary bonding nature. The adhesive composition allows for the attachment means to adhere to the metal of the flattened head.

While the invention has been described by specific examples and embodiments, there is no intent to limit the inventive concept except as set forth in the following claims.

I claim:

1. A hand-held tool useful for performing cosmetic dermabrasion comprising:
(a) a handle comprising elongated substantially cylindrical lightweight metal;
(b) a flattened head having two sides comprising the same lightweight metal as the handle;
\textcircled{c} at least one removable pad for dermabrasion, the pad having two sides, a primary side containing an abrasive material and a secondary side containing hook and loop fasteners; and
(d) an attachment means comprising a web for covering the flattened head, said attachment means having two sides, a first side containing adhesive material and a second side containing hook and loop fasteners.

2. A tool according to claim 1 wherein two removable pads are attached to the tool, one pad on each side of the flattened head.

3. A tool according to claim 1 wherein the metal handle has a diameter of about one half of an inch to about one and one half inches.

4. A tool according to claim 1 wherein the metal handle has a wall thickness of about 0.3 inches to about 1 inch.

5. A tool according to claim 1 wherein a removable cushioned covering is mounted over the handle.

6. A tool according to claim 5 wherein the removable covering overlays substantially the complete handle.

7. A tool according to claim 6 wherein the removable covering comprises a synthetic plastic material.

8. A tool according to claim 6 wherein the plastic material is a member selected from the group consisting of a vinyl polymer and an elastomeric polymer.

9. A tool according to claim 1 wherein the flattened head is substantially rectangular in shape.

10. A tool according to claim 9 wherein the substantially rectangular shaped head has dimensions of about 2 inches by about 3 and ½ inches.

11. A tool according to claim 1 wherein the abrasive material comprises silicon carbide.

12. A tool according to claim 2 wherein the two removable pads comprise pads having abrasive material of two different mesh, one pad having a coarse grade of abrasive material and a second pad having a fine grade of abrasive material.

13. A tool according to claim 1 wherein the secondary side of the removable pad contacts the second side of the attachment means.

14. A tool according to claim 1 wherein the first side of the attachment means contacts the flattened head on both sides of the head.

15. A tool according to claim 1 wherein the attachment means passes over the top of the flattened head for contact with the flattened head on both sides.

16. A tool according to claim 1 wherein the lightweight metal comprises aluminum.

17. A tool according to claim 1 wherein the flattened head is prepared by pressing about 40% of a lightweight metallic tubing with a hydraulic press, and wherein the remaining 60% of the tubing comprises the handle.

18. A method of performing cosmetic dermabrasion comprising the steps of:
(a) washing and scrubbing a part of the human anatomy with an aqueous composition comprising a dermatologically acceptable surfactant;
(b) completely drying the part of the anatomy; and
(c) performing cosmetic dermabrasion on the part of the anatomy, wherein the dermabrasion is performed with a tool comprising
(1) a handle comprising an elongated substantially cylindrical lightweight metal;
(2) a flattened head having two sides comprising the same lightweight metal as the handle; (3) at least one removable pad for dermabrasion, the pad having two sides, a primary side containing an abrasive material and a secondary side containing hook and loop fasteners; and (4) an attachment means comprising a web for covering the flattened head, said attachment means having two sides, a first side containing adhesive material and a second side containing hook and loop fasteners.

19. A method according to claim 18 wherein the part of the human anatomy is a member selected from the group consisting of the foot, the hand, the elbow, the knee and the toe.

20. A method according to claim 18 further comprising the steps of:
(d) applying lotion to the part of the human anatomy that has received dermabrasion in step (c);
(e) removing the at least one removable pad from the tool; and
(f) offering the thus removed pad to the customer who possesses the part of the anatomy so treated.
